(19) United States
(12) Patent Application Publication
(10) Pub. No.: US 2026/0007481 A1
(43) Pub. Date: Jan. 8, 2026

(54) CARDIOVASCULAR SURGICAL DEVICES, SYSTEMS, AND METHODS

(71) Applicant: Hisham BASSIOUNY, Chicago, IL (US)

(72) Inventor: Hisham BASSIOUNY, Chicago, IL (US)

(21) Appl. No.: 19/136,731

(22) PCT Filed: Jun. 4, 2024
(86) PCT No.: PCT/US2024/032430

§ 371 Date: Jun. 6, 2025

Related U.S. Application Data
(60) Provisional application No. 63/506,328, filed on Jun. 5, 2023.

(51) Int. Cl. A61B 34/30; A61F 2/958

(52) U.S. Cl. CPC A61B 34/30; A61F 2/958; A61B 2034/301; A61B 2034/306

(57) ABSTRACT

A robotic surgical method for repair of an infrarenal aortic aneurysm with an aneurysm sac in an aorta of a patient includes introducing at least one arm and a camera of a robotic surgical system into designated ports of the patient. Then, a graft is attached to a proximal wall of the aorta. The aneurysm sac is decompressed by inflating an intravascular balloon and initiating flow in an arteriovenous circuit. Anterior posterior walls of the aneurysm sac are stapled and the robotic incisions are closed. A method for inserting a conduit includes deploying an intimal umbrella, partially expanding an expandable graft, deploying an adventitial umbrella, and fully inflating the expandable stent.

### BACKGROUND

### Technical Field

The present disclosure is generally directed to cardiovascular surgical techniques and devices and systems employed in such techniques, whether performed manually or robotically assisted.

### Description of the Related Art

Each year, about 200,000 people in the U.S. are diagnosed with an abdominal aortic aneurysm (“AAA”), which is a bulge in the lower part of the main artery of the body, the aorta. The most common location of arterial aneurysm formation is in the abdominal aorta, and more specifically, the segment of the abdominal aorta below the kidneys. An abdominal aneurysm located below the kidneys is called an infrarenal aneurysm or an infrarenal abdominal aortic aneurysm (“IAAA”).

Anyone is at risk for an AAA, but there is a greater risk if you are a male age 65 years or older or have a history of smoking. AAAs are found in 2% to 8% of such patients in developed countries and can progress to a life-threatening rupture if left untreated. The mortality rate for ruptured AAAs is as high as 80%. The risk of AAA rupture is directly related to the diameter of the aneurysm. Small aneurysms (i.e., with a comparatively small diameter) have a low rupture risk and are therefore observed with serial imaging, whereas large aneurysms (i.e., with a comparatively large diameter) are prone to rupture and are repaired prophylactically given that the risk of rupture outweighs the risks of repair. Overall, AAA outcomes have improved over time due to expansion of AAA screening efforts, introduction of minimally invasive repair techniques, care coordination, and growth of patient-centered management strategies. However, these improved outcomes are not universally experienced, and disparities continue to exist by sex, race, and ethnicity.

The process for prophylactic repair of an AAA located below the kidneys (i.e., an IAAA) may be referred to as infrarenal aortic aneurysm repair (“IAAR”). IAAR is currently accomplished with three interventional techniques: minimally invasive endovascular aortic aneurysm repair (“EVAR”); open surgical repair (“OSR”); and laparoscopic surgery or robotic assisted surgery (“RAS”).

As will be explained below, each of these existing techniques have deficiencies and drawbacks with a disparity in outcomes that may be influenced by sex, race, and ethnicity, among other factors. Accordingly, it would be beneficial to have IAAR techniques that overcome the deficiencies and disadvantages of known techniques and provide more consistent outcomes for all patients.

Additional challenges can arise when introducing a conduit into the abdominal aorta to provide inflow to iliac, femoral, or visceral vessels, whether as part of the IAAR procedures, or as a standalone procedure. Specifically, aortic clamp control is required in OSR techniques, which significantly increases risk for the patient. Accordingly, it would likewise be beneficial to have techniques that overcome the deficiencies and disadvantages of known techniques related to introduction of conduits to provide more consistent outcomes for all patients.

### BRIEF SUMMARY

The present disclosure is generally directed to robotic assisted surgical techniques for the repair of an IAAA. The techniques presented herein overcome the drawbacks of conventional technologies, while maintaining many of the benefits. The technique begins by inserting an occlusion catheter into at least one common femoral artery of the aorta of the patient and positioning a proximal tip of the occlusion catheter at an aortic bifurcation to siphon residual blood from the aneurysm sac. Then, at least one arm and a camera of a robotic surgical system are introduced into designated ports of the patient. The techniques described herein continue by dividing a posterior parietal peritoneum of the patient lateral to the aorta and medial to a gonadal vein of the patient along a length of the aorta from a left renal vein to a location beyond a left common iliac artery and slinging a posterior peritoneal apron developed by medial dissection with transcutaneous monofilament sutures to retract a small bowel of the patient.

Then, the technique continues by clipping and dividing an inferior mesenteric artery and dissecting left and right iliac arteries at an intended installation location of a limb of the graft distal iliac or femoral anastomosis. Proximal aortic control is achieved with a clamp and a junction between the undiseased segment of the aorta and the aneurysm sac is stapled. A graft is then attached to a proximal wall of the aorta according to different techniques discussed herein and the aneurysm sac is decompressed by inflating an intravascular balloon and initiating flow in an arteriovenous circuit. After decompression, the anterior and posterior walls of the aneurysm sac are stapled or plicated to obliterate the sac space. The iliac balloon catheter is withdrawn to a location distal to a planned site of an iliac anastomosis and an iliac artery is occluded with sutures or staples. Finally, the occlusion catheter is withdrawn, the at least one common femoral artery puncture site is sealed with a percutaneous closure device, adequate intraperitoneal hemostasis is achieved, a posterior parietal peritoneum is approximated to isolate the graft from bowel loops, and the robotic incisions are closed.

The present disclosure also discusses techniques for introduction of a suture-less conduit into the abdominal aorta to provide inflow to iliac, femoral, or visceral vessels without aortic clamp control, which is currently unavailable in clinical practice. Such techniques are designed to limit or prevent aortic bleeding from the aortic puncture upon introduction of the conduit and thus improve the likelihood of successful outcomes.

Additional features and advantages of the techniques are provided below.

### BRIEF DESCRIPTION OF THE SEVERAL VIEWS OF THE DRAWINGS

The present disclosure will be more fully understood by reference to the following figures, which are for illustrative purposes only. These non-limiting and non-exhaustive implementations are described with reference to the following drawings, wherein like labels refer to like parts throughout the various views unless otherwise specified. The particular shapes of the elements as drawn may have been selected for ease of recognition in the drawings. The figures do not describe every aspect of the teachings disclosed herein and do not limit the scope of the claims.

FIG. 1 is a photograph array of sequential steps in an EVAR technique for an IAAA with a modular endograft.

FIG. 2 is a photograph array illustrating possible endoleaks of an IAAA after an EVAR procedure.

FIG. 3 is a photograph array of sequential steps in an OSR technique for an IAAA.

FIGS. 4A and 4B are photographs of aortoiliac exposure and proximal aortic control according to techniques of the present disclosure.

FIG. 5 is a schematic illustration of isolation of the IAAA with staples and balloon catheters according to techniques of the present disclosure.

FIG. 6 is a schematic illustration of a sutured end to side anastomotic configuration with a transverse aortotomy in the anterior aortic wall according to techniques of the present disclosure.

FIG. 7 is a schematic illustration of a hybrid graft utilized in the techniques according to the present disclosure.

FIGS. 8A and 8B are schematic illustrations of sequential steps of transaortic over the wire introduction of a sheathed hybrid stent graft according to techniques of the present disclosure.

FIGS. 9A and 9B are schematic lateral and anteroposterior illustrations, respectively, of a stapled aortic aneurysm sac according to techniques of the present disclosure.

FIG. 10 is a schematic illustration of an aortic conduit connection from the normal infrarenal aortic segment and complete aneurysm sac obliteration according to techniques of the present disclosure.

FIG. 11 is a schematic illustration of a hybrid stent utilized in the techniques according to the present disclosure in a deployed configuration.

FIG. 12 is a schematic illustration of the hybrid stent of FIG. 11 in a constrained configuration.

FIG. 13 is a schematic illustration of an adventitial umbrella utilized in the techniques according to the present disclosure.

FIG. 14 is a schematic top view of the adventitial umbrella of FIG. 13.

### DETAILED DESCRIPTION

Persons of ordinary skill in the relevant art will understand that the present disclosure is illustrative only and not in any way limiting. Other implementations of the presently disclosed systems and methods readily suggest themselves to such skilled persons having the assistance of this disclosure.

Each of the features and teachings disclosed herein can be utilized separately or in conjunction with other features and teachings to provide IAAR techniques, devices, systems, and methods. Representative examples utilizing many of these additional features and teachings, both separately and in combination, are described in further detail with reference to the attached figures. This detailed description is merely intended to teach a person of skill in the art further details for practicing aspects of the present teachings and is not intended to limit the scope of the claims. Therefore, combinations of features disclosed in the detailed description may not be necessary to practice the teachings in the broadest sense and are instead taught merely to describe particularly representative examples of the present technology.

Moreover, the various features of the representative examples and the dependent claims may be combined in ways that are not specifically and explicitly enumerated to provide additional useful implementations of the present teachings. It is also expressly noted that all value ranges or indications of groups of entities disclose every possible intermediate value or intermediate entity for the purpose of original disclosure, as well as for the purpose of restricting the claimed subject matter. The dimensions and the shapes of the components shown in the figures are designed to help understand how the present teachings are practiced but are not intended to limit the dimensions and the shapes shown in the examples in some implementations. In some implementations, the dimensions and the shapes of the components shown in the figures are exactly to scale and intended to limit the dimensions and the shapes of the components.

The present disclosure is generally directed to techniques for the repair of AAAs. The techniques of the present disclosure may be particularly advantageous for IAAR procedures but are not limited thereto. In other words, the concepts of the disclosure can be applied equally to repair of all AAAs, regardless of location along the aorta. Further, the techniques discussed herein may be suitable for application to other procedures beyond IAAR and repair of AAAs generally, such as RAS techniques in general. Thus, while the present disclosure will proceed to discuss concepts related to IAAR, it is to be appreciated that the disclosure is not limited thereto.

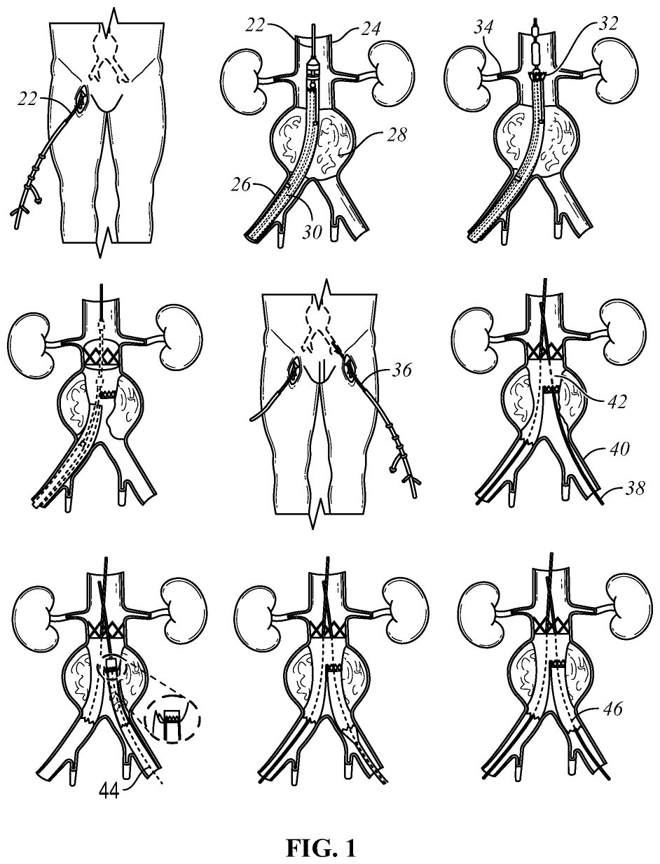

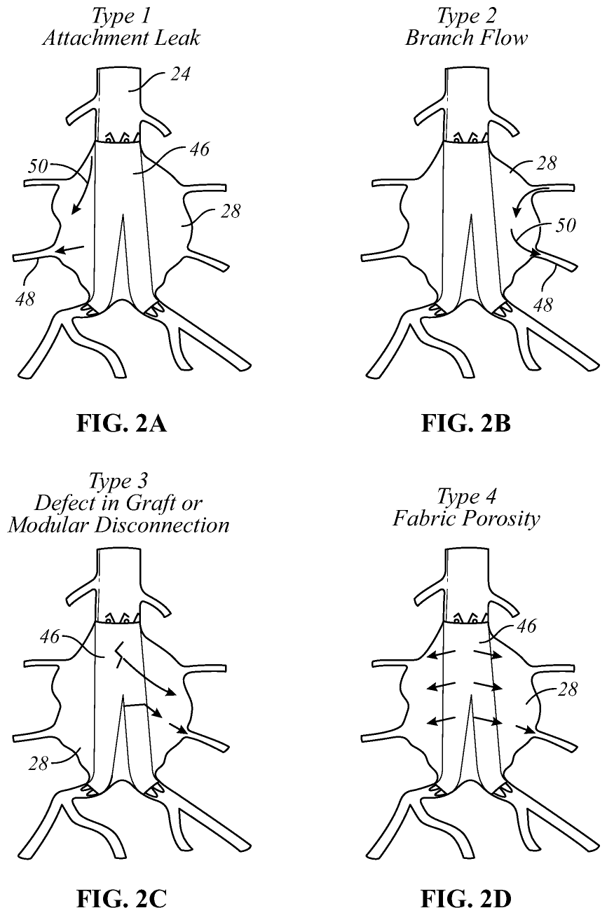

FIG. 1 is a photograph array of sequential steps in an EVAR technique for an IAAA repair with a modular endograft. FIG. 2 is a photograph array illustrating possible endoleaks of an IAAA after an EVAR procedure. FIG. 3 is a photograph array of sequential steps in an OSR technique for an IAAA. FIGS. 1-3 are provided to illustrate conventional techniques for IAAR and the associated drawbacks and disadvantages of those techniques to provide additional context regarding the concepts of the disclosure and the advantages achieved thereby.

Beginning with FIG. 1, illustrated therein is a photograph array of sequential steps in a conventional EVAR technique. EVAR using stent grafts has largely replaced OSR as the most common method worldwide for elective and emergent repair of AAAs. In recent years, the annual average number of AAA repairs is approximately 45,000 with 74% being treated with EVAR in the United States.

Nevertheless, outcomes for EVAR and OSR have been improving in select high volume centers with reports of perioperative mortality reaching rates as low as 0% and 0.5% for EVAR and OSR, respectively. EVAR is a less invasive procedure with improved short-term survival and quality of life as compared to OSR, but long-term outcomes and advantages continue to be investigated. EVAR has major drawbacks which include but are not limited to: (i) aneurysmal sac endoleaks (i.e., persistent blood flow and pressurization of the aneurysm sac); (ii) stent graft migration negating the efficacy of EVAR in excluding and depressurization of the aortic aneurysm sac; and (iii) stent graft fabric fatigue and component disconnection. Such complications are observed in at least 20% of cases and require re-interventions to prevent delayed aneurysm rupture and death. Accordingly, patients with EVAR are subject, by necessity, to long-term obligatory surveillance of the stent graft and aneurysmal sac with serial contrast-enhanced CT or duplex ultrasound imaging for the lifetime of the patient.

Systematic reviews by the U.S. Society for Vascular Surgery (“SVS”) showed a significant incidence of postoperative endoleaks up to 5 years after EVAR, which necessitates periodic image surveillance. This adds to the cumulative cost of EVAR over time and potential exposure of patients to hazardous ionizing irradiation from such imaging and surveillance. Surveillance after EVAR is performed to identify sac growth, endoleak, device migration, or device failure, among others. A comprehensive analysis of contemporary Medicare patients revealed that the incidence of late rupture 8 years after EVAR is >5%. Unfavorable anatomy for endovascular repair predisposed to most ruptures, which developed from type I or type III endoleaks with sac enlargement. Initially recommended surveillance protocols are consistent with those used by FDA-sponsored pivotal trials, with CT imaging at 1 month, 6 months, and 12 months and yearly thereafter. The 6-month CT scan can be eliminated from routine surveillance if the 1-month scan shows no concerning endoleak or sac enlargement.

Thus, the underlying factors responsible for EVAR failures are fundamentally attributed to continued presence of at least: (i) an intact aortic aneurysm sac; (ii) reliance on the radial force of the device to achieve adequate seal; and (iii) degeneration and weakening of the aortic wall leading to inadequate sealing at the proximal and distal attachment zones of the stent graft.

Another major drawback for EVAR is that surveillance noncompliance rates approach 60%, with gaps observed 3 to 4 years after EVAR, particularly among patients of advanced age, with Medicaid eligibility, or after treatment at a low-volume center. Moreover, surveillance is logistically challenging and costly in low- and middle-income countries outside of the U.S. and Europe. Although the risks of late device-related complications and aneurysm rupture are well documented, population studies have not demonstrated that annual EVAR surveillance confers a survival benefit or decreases aneurysm-related mortality. Not all late ruptures are preceded by endoleak or sac enlargement, which suggests that not all late ruptures can be prevented by vigilant surveillance. Thus, even though routine surveillance is recommended by the vascular societies, its value is questioned in preventing EVAR complications. Again, these reports strongly suggest that continued presence of an intact aneurysm sac with EVAR remains the most important denominator for EVAR complications.

FIG. 1 provides an example of steps in a known EVAR technique 20. The technique 20 begins with a guide wire 22 that is introduced into an abdominal aorta 24 through a femoral artery 26. The guide wire 22 is extended through an aneurysm sac 28 in the aorta 24. Then, a sheathed main body of a stent graft 30 is guided along the wire 22 with the use of fluoroscopy. The stent graft 30 may be composed of a non-permeable polyester material supported by a self-expanding flexible metal frame. The main body of the stent graft 30 is partially deployed, with an undeployed portion 32 of the device positioned below renal arteries 34. A second guide wire 36 is introduced through a contralateral iliac artery 40 into an open lumen 42 of the contralateral limb of the stent graft 30. The top cap of the main body of the graft 30 is removed, deploying the suprarenal stent and anchoring the main body of the stent graft 30 to the wall of the aorta 24. A contralateral limb component 44 of the stent graft 30 is then introduced via the second guide wire 36 and deployed with overlap sufficient to reduce or prevent leakage around the junction of the main body of the stent graft 30 and the proximal end of the limb 44. The ipsilateral limb of the main body of the stent graft 30 is deployed in similar fashion. A balloon is then introduced and used to expand all graft to vessel and graft to graft junctions in the proximal to distal direction to ensure a tight seal. Finally, the sheaves of the stents 30, 44 and guide wires 22, 38 are removed to complete the repair. Aortic blood subsequently flows through the installed stent graft 46.

One pronounced example of the drawbacks of EVAR procedures is the possibility of endoleaks that may occur following an EVAR procedure, such as procedure 20. FIG. 2 is a photograph array illustrating possible leak locations following an EVAR procedure. A first type of leak that may occur is an attachment leak shown in Image A of FIG. 2. With an attachment leak, blood continues to enter the aneurysm sac 28 at one of the three ends of the bifurcated stent graft 46. Specifically, blood enters at the points where the stent graft 46 should be tightly affixed to the arterial wall. Egress, as with all endoleaks, is through branches 48 of the aorta 24 that remain patent, as generally shown by arrows 50. Treatment is indicated for this first type of leak.

A second type of leak that may occur is a branch artery leak shown in Image B of FIG. 2. With a branch artery leak, blood enters the aneurysm sac 28 through a patent branch artery 48. This type of leak can be self-limited and may be just observed. Treatment is indicated if the aneurysm 28 enlarges. A third type of leak may occur with a loss of integrity of the stent graft 46, as shown in Image C of FIG. 2. Either the modules of the stent graft 46 have become separated or a rent has formed in the fabric of the stent graft 46. If so, blood enters the sac 28 from the lumen of the stent graft 46 through the site of loss of stent graft integrity. Treatment is indicated. The fourth and final type of leak may occur due to fabric porosity, as shown in Image D of FIG. 2. With this type of leak, blood enters the sac 28 from the lumen of the stent graft 46 through intact cloth of the stent graft 46. This is self-limited and present only at surgery. The pores of the fabric of the graft 46 quickly become occluded by blood products following the procedure. As described further herein, endoleaks, such as those represented in FIG. 2, can lead to hemorrhage and life-threatening rupture of the sac 28, among other serious potential outcomes.

The second known technique, OSR, is explained in more detail with reference to FIG. 3. OSR is considered the most durable intervention for abdominal aortic aneurysms by virtue of complete exclusion of the aneurysmal sac from the arterial circulation. OSR, however, has fallen into disfavor as the procedure is fraught with significant drawbacks, including at least: (a) the invasive nature of the procedure; (b) longer hospital length of stay and (c) higher 30-day morbidity and mortality ranging from 3% to 7% compared to EVAR nationwide. In an OSR procedure, such as procedure 60 shown in FIG. 3, the aorta 62 and arteries 64 are first clamped on either side of the AAA sac 66, as in Image I of FIG. 3. Then, in Image 2 and Image 3 of FIG. 3, the AAA 66 is cut open and a bifurcated prosthetic graft 68 is connected between the aorta 62 and the arteries 64. Fluid and other matter from the opened AAA sac 66 can be removed using known techniques. After the prosthetic graft 68 is sutured to the proximal normal aorta and the distal iliac or femoral arteries, the sac 66 can be closed around the graft 68 or removed, as in Image 4 of FIG. 3.

Calculating cost of care does not shed light on the benefit of care. The major benefit of treatment for patients with an aortic aneurysm (AAA) is increased survival. However, patients endure a decreased quality of life after OSR, which may be prolonged should a complication occur. Because EVAR confers a lower complication rate and smaller incisions compared with OSR techniques, patients undergoing EVAR generally have better health-related quality of life within the first 12 months, although there is no significant difference between the two techniques beyond the first year following the procedure. When cost and effectiveness are combined, cost-effectiveness analysis can reveal the value of different treatment options and allow comparison to other treatments in other fields. Early Markov decision analysis models show that EVAR is cost-effective compared with OSR techniques, with an incremental cost-effectiveness ratio of $22,826. However, contemporary Markov models using data from the DREAM, EVAR 1, OVER, and ACE randomized trials showed EVAR to be cost-effective based on the OVER trial data, but no difference in lifetime cost-effectiveness was derived from data generated by the European trials, suggesting that results may not be generalizable among different countries. EVAR also does not appear to be cost-effective for treatment of complicated aneurysms. Cost comparisons for fenestrated or branched EVAR graft demonstrated higher costs in comparison to OSR techniques (38,212 vs 16,497) without significant differences in 30-day mortality.

In view of the discussion above, it would be beneficial to have a minimally invasive procedure that mitigates EVAR drawbacks and at the same time offers the distinct advantages of OSR, namely sac exclusion and long-term durability. Recently, laparoscopic techniques have been studied that aim to achieve a more advantageous solution. However, known laparoscopic techniques likewise have drawbacks and deficiencies, as further explained below.

The third conventional technique, or laparoscopic techniques for the treatment of IAAR include a total laparoscopic approach, a laparoscopic assisted surgical approach (laparoscopic dissection with endoaneurysmorrhaphy via mini laparotomy), a hand assisted laparoscopic approach, or a RAS approach. RAS initially offered the exciting potential for a minimally invasive technique that immolates OSR. However, RAS has failed to gain widespread acceptance as a reliable technique for aortic reconstruction. Laparoscopic techniques are technically demanding and require a large or significant amount of experience in laparoscopic surgery. In a recent prospective comparative multi-center study, laparoscopic aortic surgery was associated with a significantly higher risk of death and adverse events compared with conventional OSR techniques, despite a highly experienced laparoscopic surgical team. Most recent guidelines from the European Society for Vascular Surgery (“ESVS”) and the U.S. Society for Vascular Surgery (“SVS”) judge the current procedure as risky, difficult to perform, and associated with higher risk of mortality and morbidity compared to conventional OSR techniques, even in experienced hands.

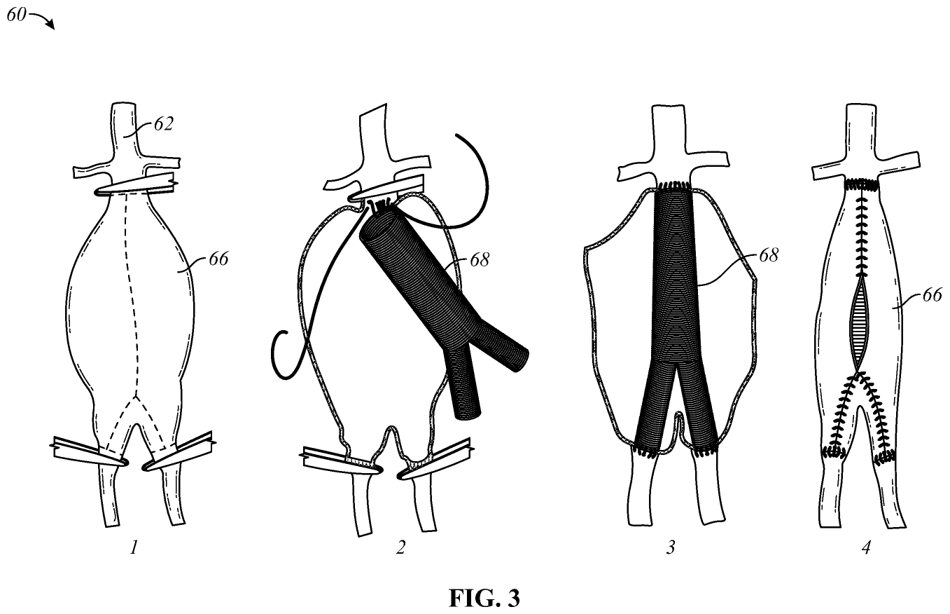

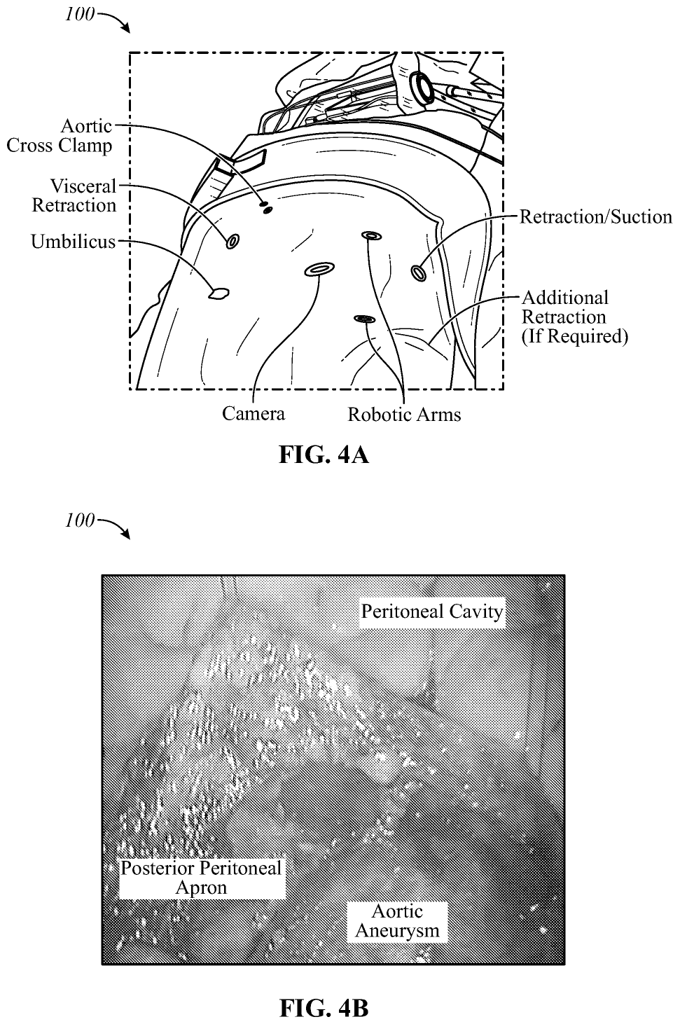

Currently, the assistance of robotic technology has proved invaluable in a variety of surgical disciplines, most notably in urological and cardiac surgery, offering improved visual acuity through high-definition, three-dimensional video monitoring of the operative field as well as allowing all possible degrees of freedom in the robotic arms during dissection and sewing. Robotically assisted vascular anastomoses can be performed faster than with a totally laparoscopic approach in reports of robotically assisted and totally robotic surgical treatment of aortoiliac occlusive disease and IAAR. The inventor of the present disclosure has previously achieved success with the laparo-robotic approach for visceral debranching followed by endovascular stent-graft placement to treat a symptomatic thoracoabdominal aneurysm in a medically high-risk patient. In other words, the inventor of the present disclosure has discovered a minimally invasive RAS technique that mitigates EVAR drawbacks and at the same time offers the distinct advantages of OSR, namely sac exclusion and long-term durability, as explained further below with reference to FIGS. 4A-10.

The purported advantage of RAS is being able to execute, in a minimally invasive fashion, the most important tenets of OSR, namely complete aneurysm sac exclusion from the arterial circulation and creating durable arterial anastomotic connection to an alternative conduit to the excluded aneurysmal aortic segment. In contrast, EVAR fails to achieve the aforementioned principal objectives as the aneurysm sac remains intact and is susceptible to possible endoleaks. Additionally, sealing of the proximal and distal stent graft attachment sites is largely radial force dependent in contrast to a sutured anastomosis with the aortic wall which is biomechanically more solid and stable. EVAR attachment site sealing inadequacies lead to endoleaks, ruptures and costly reinterventions. In recognition of these serious limitations both the SVS and ESVS mandate serial image surveillance in EVAR patients for up to 5 years following the procedure.

In utilizing technical steps that immolate those used for OSR, contemporary RAS for IAAR continues to be regarded as high risk and a technically difficult minimally invasive option for EVAR. RAS for IAAR at this moment is not considered a viable option relative to EVAR or OSR. The factors implicated for such serious RAS deficiencies in aortic repair are described in more detail below. The following description provides background regarding conventional RAS technologies and the deficiencies of the same to provide context for the benefits and advantages achieved by the techniques of the present disclosure.

In creating a proximal aortic anastomosis, a segment of normal aorta at least 2 centimeters long below the kidney arteries and outflow iliac arteries must be initially clamped to arrest blood flow to the aneurysm sac. This is followed by division of the normal aorta at its junction with the aneurysm sac, opening the sac, and sewing a prosthetic conduit to the divided normal aortic stump in an end-to-end fashion. Aortic clamping is carried out using a transabdominal Chitwood or laparoscopic clamp. Secure proximal and distal clamping below the aneurysm allows for a relatively bloodless field to precisely sew the conduit to the normal aortic stump. The technique is potentially hazardous, as respiratory excursion could result in downward slippage of the clamp and loss of aortic control leading to fatal hemorrhage. Even if slippage is observed, repositioning of the clamp or placing another clamp above the original clamp, when possible, is technically challenging and will be associated with intermittent hemorrhage or injury to the aortic wall from clamping trauma. What would have been indexed as an elective procedure then becomes a life-threatening emergency requiring open exploration to control hemorrhage and shock from a poorly clamped or injured aorta. The concepts of the disclosure describe alternative techniques to avoid and/or greatly minimize the significant risk of aortic clamping with known RAS techniques.

A principal step in IAAR either in RAS or OSR that immediately follows proximal aortic clamping is incision of the anterior wall the aneurysm sac longitudinally with cautery or scissors. The incision is carried out from its junction with the normal proximal aorta-aneurysm junction to the aortic bifurcation. At the proximal junction the aortic wall is transected in a transverse fashion (i.e., perpendicular to the longitudinal aortic axis) either completely or partially preserving the posterior aortic wall continuity to prepare the proximal aortic stump for an end-to-end anastomosis. Opening the sac is almost always associated with substantial blood loss estimated in the 500-1000 cc range and necessitates removal of any thrombus content to control retrograde bleeding from lumbar and inferior mesenteric arteries from within the aneurysm sac by oversewing. Thus, this step results in: (1) unavoidable significant blood loss; and (2) increasing aortic cross clamp time to achieve hemostasis from within the aneurysm sac. There is substantial evidence that aortic cross clamp time is a major determinant for adverse outcomes in aortic reconstruction. These include ischemic cardiac events, renal insufficiency, intestinal and lower extremity ischemia, and mortality.

From a biomechanical standpoint, creating an end-to-end anastomosis induces reduced compliance coefficient and distensibility of the aortic wall proximal to the suture line. Such biomechanical perturbations may lead to future degeneration and aneurysm formation in what was previously, prior to total or subtotal transection, a biomechanically stable aortic segment. The concepts of the disclosure provide alternative techniques to completely exclude the aneurysm sac from the arterial circulation without blood loss and greatly reduce aortic cross clamp time.

Having clamped and divided the normal aorta above the aneurysm and developed an adequate aortic stump below the proximal clamp, the prosthetic conduit selected is positioned below the aortic stump. An end-to-end anastomosis using the robotic arms is then started using an appropriate thickness, i.e., 3/0 double armed PTFE straight suture devoid of memory.

RAS is devoid of haptics and offers substantial torque strength in the robotic arms. Anastomotic suturing is carried out without a tactile sense of suture purchase and evaluation of the degree of tissue tension and approximation. This contrasts with the default direct hand technique for OSR where such tactile haptics are preserved. Suture tension and tissue approximation and deformation is thus judged by visual assessment which is arbitrary and operator dependent. To date there is no technique that would objectively measure suture tension and tissue approximation with RAS. Achieving the optimum tension is most critical, particularly when suturing the aortic wall. Excessive tension could result in injury and tearing of the aortic wall or suture material, while reduced tension can lead to gaps between the aortic stump and prosthetic conduit resulting in substantial anastomotic hemorrhage.

Because robotic arms have significant momentum, any minor lateral deviation of the curved needle could result in aortic wall tears which will bleed profusely upon unclamping. This could be a devastating event requiring re-clamping and repair of the defect with prosthetic pledgets. Aortic wall tears represent a serious and life-threatening deficiency with current RAS for IAAR. The afore-mentioned potentially life-threatening techniques and increased risk for open conversion has greatly dissuaded vascular surgeons from embracing RAS as a minimally invasive option for OSR and EVAR. The concepts of the disclosure provide alternative techniques to reconfigure the aortic graft connection with and without suturing to overcome the issues above.

In view of the above, the rationale for continued adoption of RAS in IAAR is supported by the distinct and unique capabilities of RAS which are not furnished by EVAR and OSR. Such capabilities include, but are not limited to: (I) RAS is a minimally invasive technique with 3-D visualization allowing for precise aortic dissection and control; (II) complete exclusion of the aneurysm sac is feasible hence no risk of endoleak complications that occur as with EVAR because of continued presence of an intact aneurysm sac; (III) allows access to inferior mesenteric and lumbar arteries for ligation to prevent retrograde flow in the aneurysm sac; (IV) achieves advantages of OSR, namely aneurysm sac occlusion and restoration of normal pelvic and lower extremity blood flow; and (V) a cost savings in stent graft devices.

Disruptive modifications to the current techniques are critical to establish RAS as a safe, easy to learn and ubiquitous minimally invasive interventional option for IAAR. This would require a fundamental paradigm-shift to novel techniques which would eliminate high risk RAS technical steps for IAAR as described above. Such novel and disruptive technical modifications are described in more detail below. In other words, the following description provides sequential steps in an RAS technique or techniques according to the present disclosure that overcomes the deficiencies and drawbacks of conventional IAAR intervention techniques.

### Percutaneous Transfemoral Insertion of Iliac Occlusion, Aneurysm Sac Blood Evacuation and Recirculation

An RAS technique 100 according to the present disclosure may begin with percutaneous transfemoral insertion of iliac occlusion, aneurysm sac blood evacuation, and recirculation. More specifically, a specialized 8-10 French balloon occlusion catheter is introduced percutaneously into one or both common femoral arteries as dictated by luminal diameter and severity of occlusive or aneurysmal disease of the common, external and internal iliac arteries. The proximal tip of the catheter(s) is positioned at the aortic bifurcation to siphon residual blood from the aneurysm sac. Decompression of the sac following proximal aortic control facilitates subsequent sac obliteration. The catheter balloon is inflated immediately prior to aneurysm sac exclusion and obliteration. The benefits and advantages of this approach are at least to: (a) decompress the sac and facilitate sac obliteration; and (b) achieve proximal intravascular iliac artery control while performing the distal graft anastomoses to the common or external iliac arteries.

The catheters are connected externally via a Y connector to a common tubing and the blood is returned to the common femoral vein with a percutaneously inserted 10 French sheath. By virtue of the steep pressure gradient between the arterial and venous side, the system operates as a high flow arteriovenous shunt. Blood flow in the shunt is increased or decreased by a flow controller filter which traps any micro or macro debris greater than 200 micron released from the aneurysm sac following proximal aortic control.

### Aortoiliac Exposure and Proximal Aortic Control

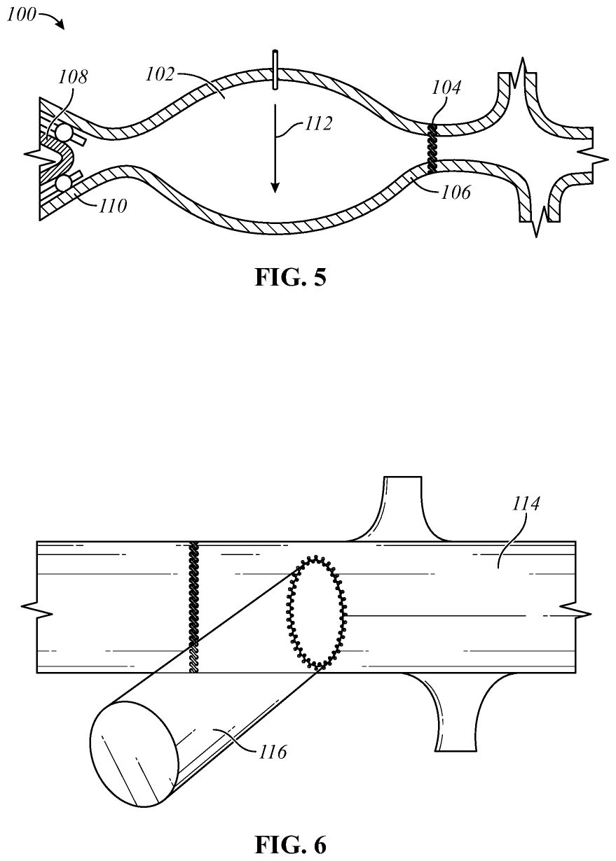

FIGS. 4A and 4B are photographs of aortoiliac exposure and proximal aortic control according to techniques of the present disclosure. FIG. 5 is a schematic illustration of isolation of the AAA with staples and balloon catheters according to techniques of the present disclosure.

After general anesthesia, the technique 100 continues with the patient positioned supine with a bump to raise the left flank and the patient is rotated to the right lateral decubitus position. After abdominal cavity insufflation the robotic system is brought adjacent to the patient, and the arms and camera are introduced through the designated ports indicated in FIG. 4A. In general, FIG. 4A identifies various ports used throughout the technique 100, including at least with respect to introduction of the aortic cross clamp, retraction and/or suction, additional retraction (optional; if required), robotic arms, camera, umbilicus, and visceral retraction.

Using the robotic bipolar grasper and monopolar scissors, the posterior parietal peritoneum was divided lateral to the aorta and medial to the gonadal vein along the length of the aorta from the left renal vein to a point beyond the left common iliac artery. A posterior peritoneal apron was developed by medial dissection above the AAA sac and slung with two transcutaneous 4-0 monofilament sutures on Keith needles to retract the small bowel, as shown in FIG. 4B. The inferior mesenteric artery is double-clipped and divided, and any visualized left lumbar arteries occluded with appropriate size clips. A sufficient retro-aortic space developed to clamp the aorta immediately below the lowest renal artery is planned. The left and right iliac arteries are dissected at the desired location for the graft limb(s) distal iliac or femoral anastomosis. After adequate anticoagulation, proximal aortic control is achieved using a laparoscopic or a Chitwood clamp introduced through a stab incision 1 centimeter (“cm”) below the xiphoid process. The normal aorta-aneurysm junction is stapled. For example, with reference to FIG. 5, the junction may be stapled with a 4.5 cm adjustable vascular stapler, such as of the type manufactured by Ethicon located in Somerville, NJ. Attention is then aortic directed to connection of the proximal aortic graft segment to the desired conduit.

In more detail, FIG. 5 illustrates isolation of an IAAA 102 proximally with a transverse staple line 104 in an aortic neck 106 just above the IAAA 102 according to technique 100. The staples may be deployed with an adjustable vascular stapler, as above. Occlusion balloon catheters 108 distal vesting orifice are inserted percutaneously via femoral arteries 110. Arrow 112 represents a direction of stapling to approximate anterior and posterior aneurysm sac walls according to technique 100.

### Creation of Proximal Aorta Graft Connection

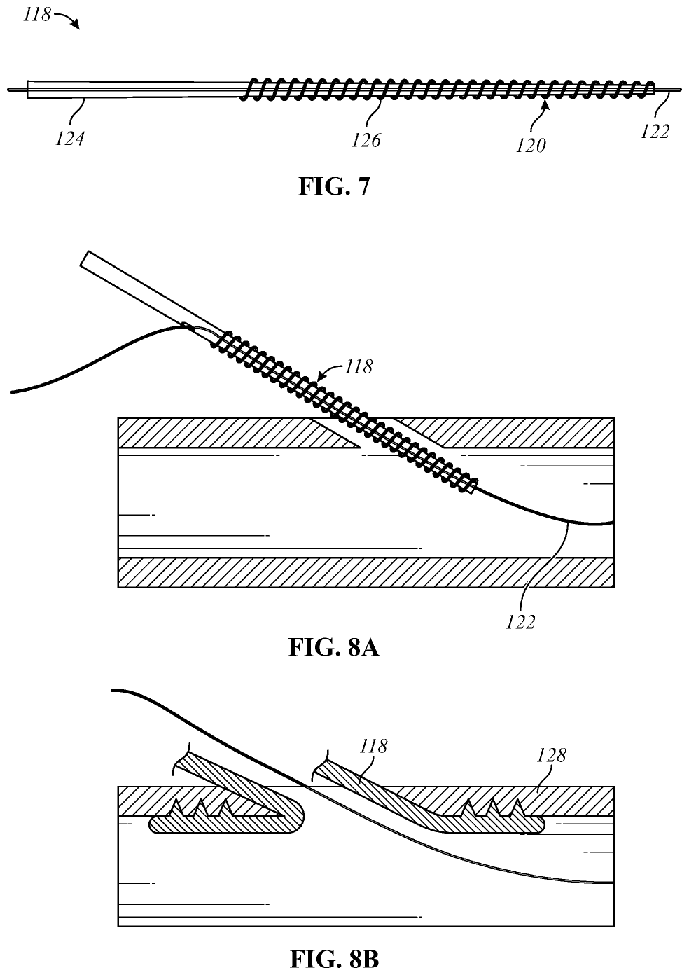

The technique 100 continues with the creation of a proximal aorta graft connection. At least two different techniques for such a connection are considered. FIG. 6 is a schematic illustration of a sutured end to side anastomotic configuration with a transverse aortotomy in the anterior aortic wall according to a first technique for proximal aorta graft connection. FIG. 7 is a schematic illustration of a hybrid graft, and FIGS. 8A and 8B are schematic illustrations of sequential steps of transaortic over the wire introduction of a sheathed hybrid stent graft according to a second technique for proximal aorta graft connection.

Beginning with FIG. 6 and the first technique, a novel anastomotic configuration with infrarenal aortic cross clamping is proposed. In contrast to customary OSR and current RAS for IAAR, end to end anastomosis is performed in the technique 100 between the proximal aortic stump and designated graft conduit (straight tube or bifurcated). The technique 100 involves creating a transverse elliptical aortotomy in the anterior aortic wall 114 while maintaining the integrity of the lateral and posterior aortic walls. An aortic side to end graft is then completed using a running 3/0 or 4/0 non absorbable knotless tissue 116 to anchor the suture to the aortic wall 114 without an assistant follow through technique.

This technique mitigates the risks attendant with the customary end to end anastomosis used in conventional OSR for at least the following reasons: (i) the anterior aortic wall is known to be spared of atherosclerosis, is more pliable, and more biomechanically solid structure than the posterior aortic wall with the latter being the most frequent site for anastomotic suture line tears and significant hemorrhage requiring extended aortic cross clamping and hemodynamic instability; (ii) more expedient and substantially easier to perform the anastomosis and to correct any suture line defects involving the anterior aortic wall because of ease of visualization and access to suturing; (iii) avoids technical difficulties encountered when sewing or repairing a suture line involving a potentially thin and/or atherosclerotic plaque ridden posterior aortic wall; (iv) allows for including a broad rim of the aortic wall beyond the elliptical aortotomy by the suture needle, which accomplishes exceptional strength for the suture line and greatly minimizes any risk for anastomotic leaks and hemorrhage; (v) the functionality of this novel anastomotic configuration is like an end-to-end anastomosis due to presence of a stapled distal aortic stump; and (vi) creation of a transverse elliptical aortotomy reduces the length of the infrarenal aortic stump needed to complete the anastomosis, which facilitates creation of the anastomosis at the juxta-renal aorta if necessary. In contrast, a typical end to end anastomosis requires at least a 2 cm cm normal aortic segment to ensure adequate suturing of the posterior aortic wall.

Upon completion of the sutured aorta to graft anastomosis, the clamp is temporarily released to flush the graft 116 and the graft 116 is clamped distal to the suture line. After completing the connection of the proximal aortic segment, iliac balloons may then be inflated to arrest retrograde flow into the aneurysm sac 102 in some implementations.

Turning to FIG. 7 and FIGS. 8A and 8B, illustrated therein are concepts associated with transaortic graft implantation without infrarenal aortic cross clamping according to the second technique referenced above.

In this technique, a specifically designed hybrid prosthetic PTFE or Dacron graft 118 (8-12 mm transverse diameter) is developed and includes a constrained covered Nitinol expanded proximal segment 120.

This hybrid graft 118 is introduced over a 0.035-inch stiff wire 122 into aortic lumen via an 18-gauge needle followed by a hybrid graft introducer sheath 124. The constrained hybrid graft 118 is introduced into the sheath 124, which is withdrawn exposing a graft marker 15 mm from the intraluminal proximal tip of the graft 118. The marker is adjusted at the level of the aortic wall. The Nitinol stent constraining wire 126 is released such that the Nitinol covered graft 118 assumes a T-shaped configuration. The graft 118 is withdrawn to a designated marker on the balloon expansible stent until the horizontal Nitinol annulus opposes the luminal side of the aortic wall. The adventitial umbrella in introduced over the stent graft, deployed and stapled to the adventitial side of the aortic wall, then the balloon expandible stent 118 at its junction with the aortic wall is gradually dilated with semi-compliant balloons until the nominal size of the covered stent is reached. The distal unstented segment of the hybrid graft 118 is unclamped and flushed. The adventitial umbrella of the hybrid graft is anchored to the anterior aortic wall with 4 interrupted 4/0 prolene sutures.

In particular, FIG. 8A illustrates a transaortic introduction of the sheathed hybrid stent graft 118 over the wire 122 according to the process described above. FIG. 8B illustrates the unsheathed T-shaped configuration of the expanded hybrid stent graft 118 secured to the aortic wall 128. Among other benefits, the concepts of FIG. 8A and FIG. 8B eliminate the need for aortic cross clamping, thus reducing or eliminating the risks associated with aortic cross clamping described herein.

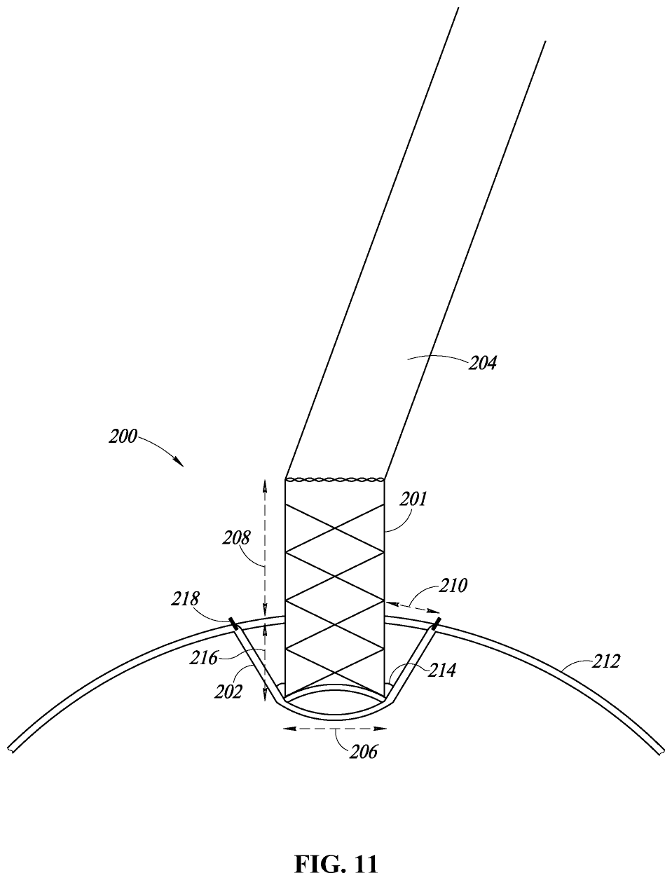

In an embodiment, FIG. 8A and FIG. 8B and the corresponding description provide one non-limiting proposed solution for transaortic graft implantation without infrarenal aortic cross clamping. The disclosure also contemplates an additional proposed solution that is discussed further with reference to FIGS. 11-14.

### Aneurysm Sac Exclusion and Obliteration

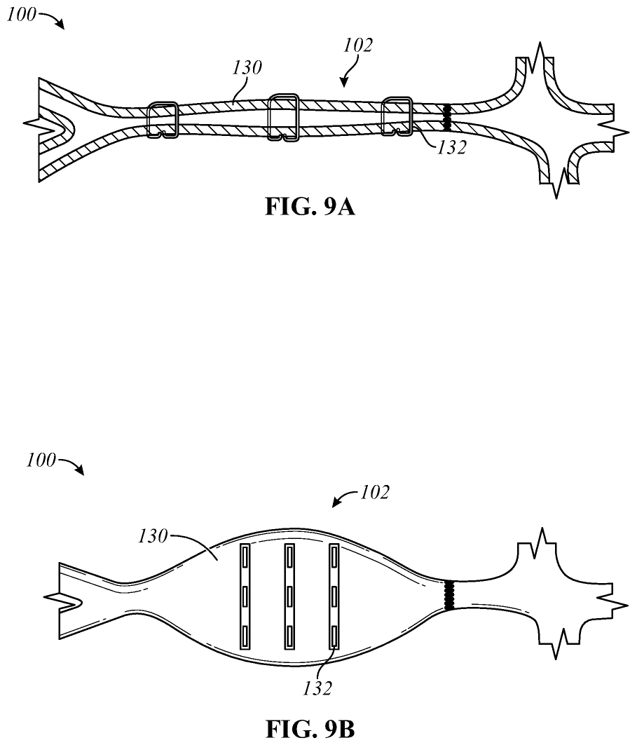

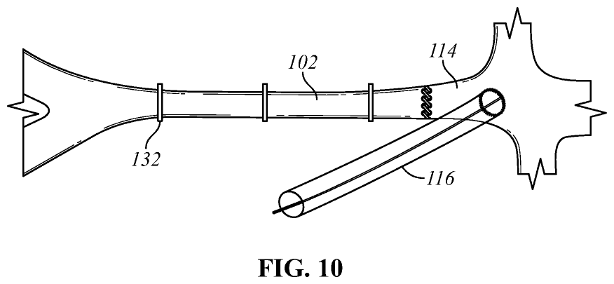

FIGS. 9A and 9B are schematic illustrations of lateral and anteroposterior views of a stapled aortic aneurysm sac according to techniques of the present disclosure. FIG. 10 is a schematic illustration of an aortic conduit connection from the normal infrarenal aortic segment and complete aneurysm sac obliteration according to techniques of the present disclosure.

With reference to FIGS. 9A-10, once the proximal aorta graft connection is established, an iliac intravascular balloon is inflated and flow is initiated in the arteriovenous circuit to decompress the aneurysm sac. Using a newly designed stapler, the anterior and posterior sac walls are closely approximated by 3-5 transverse staple rows or can be obliterated with trifoliate shaped 1-0 Prolene sutures on a large 7.5 cm CTX-B curved cutting needle, such as of the type made by Ethicon.

FIG. 9A provides a non-limiting example of a lateral view of a stapled aortic aneurysm sac according to the above techniques. As shown in FIG. 9A, walls 130 of the aneurysm sac 102 are closely approximated to each other by staples 132. The staples 132 may be arranged in transverse rows, such as between 3 to 5 rows. FIG. 9B is an anteroposterior view of the stapled aortic aneurysm sac 102 that more clearly illustrates the rows of staples 132. The number of staples 132 in each row may generally be selected based on a number of factors described herein as well as the characteristics of the aneurysm sac 102, with the three staples 132 per row provided only as a non-limiting example.

FIG. 10 illustrates an aortic conduit connection from the normal infrarenal aortic segment and complete aneurysm sac obliteration. In sum, FIG. 10 illustrates a combination of the above steps where the graft 116 is attached to the anterior aortic wall 114 and the aneurysm sac 102 is stapled to provide an aortic conduit connection from the normal infrarenal aortic segment (i.e., aortic walls 114) and complete obliteration of the aneurysm sac 102.

### Distal Anastomosis

Following the above steps, the iliac balloon catheter is withdrawn to a location distal to the planned site of the iliac anastomosis, as determined by a retrograde iliac arteriogram. The balloon is inflated to achieve retrograde blood flow control. Proximal to the iliac anastomotic site the iliac artery is permanently occluded with transfixtion 4/0 sutures or an appropriate size stapler. The anastomosis is created in and end to side fashion using 4 or 5/0 prolene sutures. Prior to restoration of distal arterial flow to the pelvis and lower extremities, adequate fore and back bleeding at the anastomotic site is performed prior to full completion of the suture line. The balloon catheters are then removed, and the common femoral artery puncture site sealed using conventional techniques, such as with percutaneous closure devices.

### Procedure Termination

After achieving adequate intraperitoneal hemostasis, the posterior parietal peritoneum is then approximated with 3/0 vicryl to isolate the graft from any bowel loops and the robotic incisions are closed with interrupted sutures.

### Trans-Aortic Arterial Suture-Less Conduit (“TASC”)

To date, a suture-less conduit that can be introduced into the abdominal aorta to provide inflow to iliac, femoral, or visceral vessels without aortic clamp control is unavailable in clinical practice. As referenced above, the use of aortic clamp control increases the risk to the patient due to interruption of blood flow to vital organs and the possibility of aortic wall trauma by application of the clamps. Furthermore, creating a sutured conduit to the aortic wall is fraught with risk of hemorrhage from the suture line.

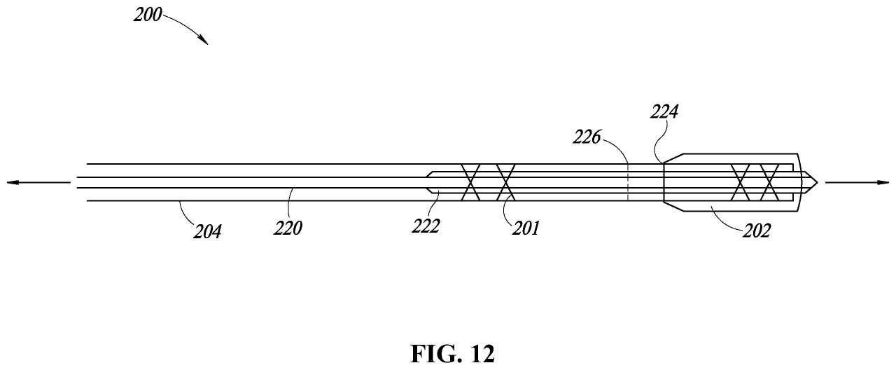

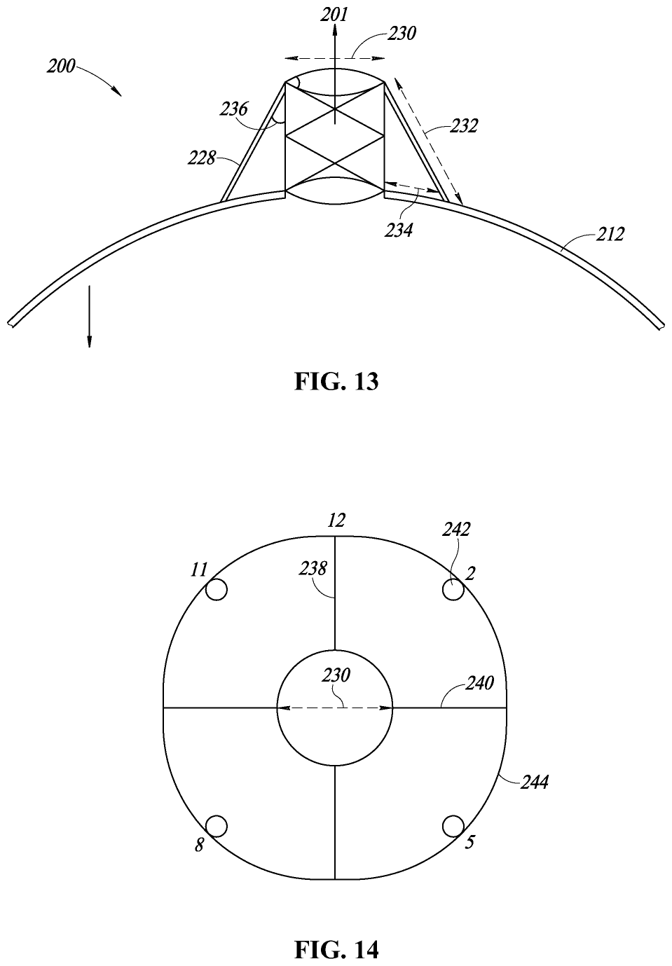

The embodiments of the disclosure provide for such a suture-less conduit to meet this long-felt need in the market. The conduit is designed for application during robotic assisted and open (manual) vascular surgical procedures. The conduit includes two principal components which can be deployed upon exposure of the selected aortic segment deemed suitable for providing adequate conduit arterial inflow. The first principal component (or primary component) is a hybrid stent graft (FIG. 11) consisting of an intimal umbrella and a balloon expandable stent graft in continuity with a conventional polytetrafluoroethylene graft (FIG. 12). In an embodiment, the stent graft can be designed with or include branches. A second principal component (or secondary component) of the conduit is an adventitial umbrella (FIG. 13 and FIG. 14). The intimal and adventitial umbrella are designed to prevent aortic bleeding from the aortic puncture upon introduction of the conduit. The TASC techniques and devices and systems used in such techniques that are described below provide an additional or alternative solution for transaortic graft implantation without infrarenal aortic cross clamping that is introduced above with reference to FIG. 8A and FIG. 8B. Further, the TASC techniques can be used as part of an IAAR procedure, as part of other procedures, or as a stand-alone procedure. Accordingly, the TASC techniques discussed below may be used in combination with, or separately, from any of the other techniques discussed herein.

Starting with FIG. 11, illustrated therein is a conduit 200 in a deployed configuration that will be described further below. The conduit 200 includes a surgical grade stainless steel stent lined with a fluoropolymer graft 201 as well as an intimal umbrella with a nitinol lined fluoropolymer membrane 202. The intimal umbrella 202 may have a radius of 10 mm or about 10 mm, or more or less. The conduit 200 further includes a thin-walled fluoropolymer graft 204. In an embodiment, the stent 201 may have a diameter of 12 mm or about 12 mm, or more or less, as indicated by dimension 206. In an embodiment, the conduit 200 includes an extra-aortic stent 208 have a length of 15 mm or about 15 mm, or more or less. The stent 201 may be spaced by a dimension 210 from an aortic wall 212 of 5 mm or about 5 mm, or more or less. In other words, dimension 210 is the intra-aortic wall umbrella coverage distance from an outer edge of the stent 201. Further, the stent 201 and the umbrella 202 may cooperate to define an angle 214 of approximately 35 degrees. In other words, the angle 214 is an incident angle of the intimal umbrella 202 relative to the stent 201. The conduit 200 may further include an intra-aortic stent 216 having a length of 8 mm or about 8 mm, or more or less. In an embodiment, an umbrella barb 218 of the intimal umbrella 202 protrudes from the aortic wall 212.

FIG. 12 illustrates the conduit 200 in a constrained configuration that will be described further below. The conduit 200 is introduced by assistance of a balloon catheter 220 and a semi-compliant balloon 222. In an embodiment, the semi-compliant balloon 222 is a 28 mm long×12 mm wide (or diameter) balloon 222, or more or less with the semi-balloon 222 positioned around the catheter 220. The stent 201 is placed over the combination of the catheter 220 and the semi-compliant balloon 222 with the intimal umbrella 202 at an end thereof. The conduit 200 may have markings to assist with installation, such as marking 224 corresponding to a 1 cm mark on the stent 201 and marking 226 corresponding to a 1.5 cm mark on the stent 201. The stent 201 is associated with the thin-walled fluoropolymer graft 204 described above with reference to FIG. 11.

FIG. 13 illustrates the secondary component, or adventitial umbrella 228, of the conduit 200 in a deployed configuration. As will be described below, the adventitial umbrella 228 is installed after insertion of the stent 201. The stent 201 may have a diameter 230 after insertion of about 13 mm. The umbrella 228 may have a radius 232 of about 1 cm and a coverage distance 234 of the adventitial umbrella 228 may be about 5 mm. In other words, the coverage distance 234 may be the extra-aortic wall umbrella coverage distance from an outer edge of the stent 201 to the aortic wall 212. The adventitial umbrella 228 may be at an angle 236 relative to the stent 201 of about 35 degrees.

FIG. 14 illustrates a top view of the adventitial umbrella 228 of FIG. 13. In an embodiment, the conduit 200 may further include a nitinol strut 238 with a length of 10 mm or about 10 mm, or more or less. The conduit 200 may include four such struts 238, or more or less, at 90-degree internals or any other selected arrangement. The conduit 200 and associated techniques further include a fluoropolymer membrane 240 between struts 238 as well as apertures 242 for staples and a nitinol ring 244 surrounding and connected to the struts 238 and membranes 240. The apertures 242 may include four apertures 242 arranged at locations 2, 5, 8, and 11 on a 12-hour clock layout corresponding to the nitinol ring 144.

In view of the above, and with continuing reference to FIGS. 11-14, a deployment technique for the conduit 200 (or TASC 200) and associated structures will now be described. The process may begin by introducing an 18 g needle with robotic arms or manually (in open surgery) in the non- or minimally diseased aortic segment 15 mm above the proximal extent of the aortic aneurysm or diseased aortic segment.

Then, a 0.032-inch standard PTFE wire is introduced through the needle, which is then withdrawn, and the wire is exchanged via a 5 Fr multipurpose arteriographic catheter with an 0.035 Amplatz or Lunderquist catheter through a 6 French sheath. Subsequently, 10 Fr 50-60 cm straight introducer sheath marked 1.5 cm from tip is introduced into lumen. The introducer is removed, back-bleeding confirmed, and the sheath flushed with heparinized saline.

The TASC 200 is then introduced into the sheath to the marking 226 on balloon catheter 220. This indicates that the principal end of the hybrid stent 201 is at the intraluminal sheath orifice. The sheath is withdrawn 1 cm to deploy the intraluminal intimal umbrella 202 followed by the balloon expandable stent 201 which is partially inflated to 3 mm in diameter.

The 10 Fr sheath is withdrawn, and the adventitial umbrella 228 is introduced over the balloon catheter 220. Once in the abdominal cavity, the adventitial umbrella wrap is released with a draw string to deploy the umbrella 228. The umbrella cylinder is advanced using a screw on/off pusher until the umbrella 228 abuts the aortic adventitia.

After the sheath is withdrawn, 4 staples are applied to apertures at 2, 5, 8 and 11 o'clock and the balloon expandable stent 201 is inflated to 12-13 mm, such as by using semi-compliant balloon 222. The balloon catheter 220 is then removed. The final step is to pull the constraining suture of non-stented graft segment and check inflow followed by clamping graft 1 cm beyond balloon expandable stent 201.

In view of the above, the implementations of the technique 100 have a number of advantages over conventional RAS for IAAR, including, without limitation: (1) preserves the durable advantages of OSR in a minimally invasive fashion; (2) accomplishes complete aneurysm sac exclusion in contrast to EVAR where the sac is present and is susceptible to endoleaks and life-threatening rupture (i.e., eliminates risk of endoleaks and reduces or eliminates risk of life-threatening rupture); (3) the techniques simplify learning and adoption of RAS in IAAR; (4) by virtue of the anastomotic techniques described, the procedure greatly minimizes potentially harmful risks with current RAS techniques in IAAR; (5) application of novel design of a trans-vascular graft, staplers, and iliac artery balloon catheters greatly reduces intervention time; (7) collectively, there is reduced risk of repeated aortic clamping, suture line hemorrhage and extensive dissection of iliac arteries; (8) minimal periodic postoperative image surveillance is needed as dictated by international guidelines for EVAR; and (9) significant savings in stent graft costs. Further, the TASC techniques described above eliminate aortic clamp control, which provides the advantages discussed above.

One or more implementations of a robotic surgical method for repair of an infrarenal aortic aneurysm with an aneurysm sac in an aorta of a patient may be summarized as including: introducing at least one arm and a camera of a robotic surgical system into designated ports of the patient; attaching a graft to a proximal wall of the aorta; decompressing the aneurysm sac by inflating an intravascular balloon and initiating flow in an arteriovenous circuit; stapling anterior and posterior walls of the aneurysm sac; and closing the robotic incisions.

The method may further include, before the introducing the at least one arm and the camera of the robotic surgical system: inserting an occlusion catheter into at least one common femoral artery of the aorta of the patient; and positioning a proximal tip of the occlusion catheter at an aortic bifurcation to siphon residual blood from the aneurysm sac.

The method may further include, after the introducing the at least one arm and the camera of the robotic surgical system: dividing a posterior parietal peritoneum of the patient lateral to the aorta and medial to a gonadal vein of the patient along a length of the aorta from a left renal vein to a location beyond a left common iliac artery; and slinging a posterior peritoneal apron developed by medial dissection with transcutaneous monofilament sutures to retract a small bowel of the patient.

The method may further include, after the slinging: clipping and dividing an inferior mesenteric artery; and dissecting left and right iliac arteries at an intended installation location of a limb of the graft distal iliac or femoral anastomosis.

The method may further include, before the attaching the graft: achieving proximal aortic control with a clamp; stapling a junction between the aorta and the aneurysm sac; and inflating iliac balloons to arrest retrograde flow into the aneurysm sac.

The method may further include, before the closing the robotic incisions: withdrawing the iliac balloon catheter to a location distal to a planned site of an iliac anastomosis; and occluding an iliac artery with sutures or staples.

The method may further include, before the closing the robotic incisions: withdrawing the occlusion catheter; sealing the at least one common femoral artery puncture site with a percutaneous closure device; achieving adequate intraperitoneal hemostasis; and approximating a posterior parietal peritoneum to isolate the graft from bowel loops.

The method may further include the attaching the graft to the proximal wall of the aorta including attaching the graft to the aorta with a sutured end to side anastomotic configuration with a transverse aortotomy in an anterior wall of the aorta.

The method may further include the attaching the graft to the proximal wall of the aorta including introducing a sheathed hybrid stent graft via a wire.

One or more implementations of a device may be summarized as including: a sheath; a hybrid graft contained within the sheath and including a constrained covered Nitinol expanded proximal segment; and a wire, wherein the hybrid graft is configured to be introduced to an aorta of a patient over the wire and the sheath withdrawn such that the hybrid graft assumes a T-shaped configuration.

The above description of illustrated implementations, including what is described in the Abstract, is not intended to be exhaustive or to limit the implementations to the precise forms disclosed. Although specific implementations of and examples are described herein for illustrative purposes, various equivalent modifications can be made without departing from the spirit and scope of the disclosure, as will be recognized by those skilled in the relevant art. The teachings provided herein of the various implementations can be applied outside of the lift or floating lift context, and are not limited to the example lift devices, systems, methods, and devices generally described above.

Many of the methods described herein can be performed with variations. For example, many of the methods may include additional acts, omit some acts, and/or perform acts in a different order than as illustrated or described.

In the above description, certain specific details are set forth in order to provide a thorough understanding of various implementations of the disclosure. However, one skilled in the art will understand that the disclosure may be practiced without these specific details. In other instances, well-known structures associated with lift devices, systems, and methods have not been described in detail to avoid unnecessarily obscuring the descriptions of the implementations of the present disclosure.

Certain words and phrases used in the specification are set forth as follows. As used throughout this document, including the claims, the singular form “a”, “an”, and “the” include plural references unless indicated otherwise. Any of the features and elements described herein may be singular, e.g., a lift may refer to one lift. The terms “include” and “comprise,” as well as derivatives thereof, mean inclusion without limitation. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. Other definitions of certain words and phrases are provided throughout this disclosure.

The use of ordinals such as first, second, third, etc., does not necessarily imply a ranked sense of order, but rather may only distinguish between multiple instances of an act or a similar structure or material.

Throughout the specification, claims, and drawings, the following terms take the meaning explicitly associated herein, unless the context clearly dictates otherwise. The term “herein” refers to the specification, claims, and drawings associated with the current application. The phrases “in one implementation,” “in another implementation,” “in various implementations,” “in some implementations,” “in other implementations,” and other derivatives thereof refer to one or more features, structures, functions, limitations, or characteristics of the present disclosure, and are not limited to the same or different implementations unless the context clearly dictates otherwise. As used herein, the term “or” is an inclusive “or” operator and is equivalent to the phrases “A or B, or both” or “A or B or C, or any combination thereof,” and lists with additional elements are similarly treated.

Generally, unless otherwise indicated, the materials for making the invention and/or its components may be selected from appropriate materials such as composite materials, ceramics, plastics, metal, polymers, thermoplastics, elastomers, plastic compounds, and the like, either alone or in any combination.

The foregoing description, for purposes of explanation, uses specific nomenclature and formula to provide a thorough understanding of the disclosed implementations. It should be apparent to those of skill in the art that the specific details are not required in order to practice the invention. The implementations have been chosen and described to best explain the principles of the disclosed implementations and its practical application, thereby enabling others of skill in the art to utilize the disclosed implementations, and various implementations with various modifications as are suited to the particular use contemplated. Thus, the foregoing disclosure is not intended to be exhaustive or to limit the invention to the precise forms disclosed, and those of skill in the art recognize that many modifications and variations are possible in view of the above teachings.

The terms “top,” “bottom,” “upper,” “lower,” “up,” “down,” “above,” “below,” “left,” “right,” and other like derivatives take their common meaning as directions or positional indicators, such as, for example, gravity pulls objects down and left refers to a direction that is to the west when facing north in a Cardinal direction scheme. These terms are not limiting with respect to the possible orientations explicitly disclosed, implicitly disclosed, or inherently disclosed in the present disclosure and unless the context clearly dictates otherwise, any of the aspects of the implementations of the disclosure can be arranged in any orientation.

As used herein, the term “substantially” is construed to include an ordinary error range or manufacturing tolerance due to slight differences and variations in manufacturing. Unless the context clearly dictates otherwise, relative terms such as “approximately,” “substantially,” and other derivatives, when used to describe a value, amount, quantity, or dimension, generally refer to a value, amount, quantity, or dimension that is within plus or minus 5% of the stated value, amount, quantity, or dimension. It is to be further understood that any specific dimensions of components or features provided herein are for illustrative purposes only with reference to the various implementations described herein, and as such, it is expressly contemplated in the present disclosure to include dimensions that are more or less than the dimensions stated, unless the context clearly dictates otherwise.

The present application claims priority to U.S. Provisional Patent Application No. 63/506,328 filed Jun. 5, 2023, the entire contents and disclosure of which are incorporated herein by reference.

These and other changes can be made to the implementations considering the above-detailed description. In general, in the following claims, the terms used should not be construed to limit the claims to the specific implementations disclosed in the specification and the claims but should be construed to include all possible implementations along with the full scope of equivalents to which such claims are entitled. Accordingly, the breadth and scope of a disclosed implementation should not be limited by any of the above-described implementations but should be defined only in accordance with the following claims and their equivalents.

1. A robotic surgical method for repair of an infrarenal aortic aneurysm with an aneurysm sac in an aorta of a patient, comprising:
introducing at least one arm and a camera of a robotic surgical system into designated incisions in the patient;
attaching a graft to a proximal wall of the aorta;
decompressing the aneurysm sac by inflating an intravascular balloon and initiating flow in an arteriovenous circuit;
stapling or suturing the anterior and posterior walls of the aneurysm sac; and
closing the incisions.

2.-8. (canceled)

9. The method of claim 1, wherein the attaching the graft to the proximal wall of the aorta includes introducing a sheathed hybrid stent graft via a wire.

10. The method of claim 1, wherein the attaching the graft to the proximal wall of the aorta includes:
inserting a suture less conduit and a balloon catheter through a sheath in the patient;
deploying an intimal umbrella and an expandable stent of the suture less conduit into the aortic wall of the patient, including partially withdrawing the sheath to deploy the intimal umbrella and partially inflating the expandable stent;
fully removing the sheath;
introducing an adventitial umbrella over the balloon catheter and deploying the adventitial umbrella;
fully inflating the expandable stent; and
removing the balloon catheter.

11.-16. (canceled)

17. A surgical method for introduction of a suture-less conduit in an aortic wall of a patient, comprising:
inserting a suture less conduit and a balloon catheter through a sheath in the patient;
deploying an intimal umbrella and an expandable stent of the suture less conduit into the aortic wall of the patient, including partially withdrawing the sheath to deploy the intimal umbrella and partially inflating the expandable stent;
fully removing the sheath;
introducing an adventitial umbrella over the balloon catheter and deploying the adventitial umbrella;
fully inflating the expandable stent; and
removing the balloon catheter.

18. The method of claim 17, further comprising, before inserting the suture less conduit:
introducing a needle with robotic arms in a minimally diseased segment of the aortic wall;
introducing a wire through the needle and withdrawing the needle; and
exchanging the wire for the balloon catheter.

19. (canceled)

20. The method of claim 17, wherein the partially inflating the expandable stent includes inflating the expandable stent to about 3 mm in diameter.

21. The method of claim 20, wherein the fully inflating the expandable stent includes inflating the expandable stent to about 12-13 mm in diameter.

22. The method of claim 17, wherein the deploying the adventitial umbrella includes releasing a wrap on the adventitial umbrella with a draw string and advancing an umbrella cylinder with a pusher until the adventitial umbrella abuts an adventitia of the aortic wall.

23. The method of claim 17, further comprising, before fully inflating the expandable stent:
applying staples to apertures in the patient.

24. (canceled)

25. (canceled)

26. A surgical method for introduction of a suture less conduit in an aortic wall of a patient, comprising:
inserting a suture less conduit and a balloon catheter through a sheath in an aperture in the patient;
deploying an intimal umbrella and an expandable stent of the suture less conduit into the aortic wall of the patient;
introducing an adventitial umbrella over the balloon catheter and deploying the adventitial umbrella;
inflating the expandable stent;
removing the balloon catheter; and
closing the aperture in the patient.

27. The method of claim 26, wherein the deploying the intimal umbrella includes partially withdrawing the sheath to deploy the intimal umbrella, and
wherein the deploying the expandable stent includes partially inflating the expandable stent at a first instance and the inflating the expandable stent includes fully inflating the expandable stent at a second instance.

28. (canceled)

29. (canceled)

30. The method of claim 29, wherein an incident angle between the adventitial umbrella and the expandable stent is about 35 degrees.

31. The method of claim 30, wherein the intimal umbrella and the adventitial umbrella are on opposite sides of the expandable stent.

32. The method of claim 26, wherein an extra-aortic wall umbrella coverage distance of the adventitial umbrella is about 5 mm.

33. The method of claim 26, wherein an intra-aortic wall umbrella coverage distance of the intimal umbrella is about 5 mm.

34. (canceled)

35. (canceled)
